(19) United States
(12) Patent Application Publication
(10) Pub. No.: US 2004/0242569 A1
(43) Pub. Date: Dec. 2, 2004

(54) Quatemary ammonium compounds

(75) Inventors: Patrick James Lennon, Webster Groves, MO (US); Dominique Francoise Bonafoux, St. Louis, MO (US); Sergey Gregory Wolfson, Chesterfield, MO (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/823,965

(22) Filed: Apr. 13, 2004

Related U.S. Application Data
(60) Provisional application No. 60/462,956, filed on Apr. 15, 2003.

(51) Int. Cl. A61K 31/549

(52) U.S. Cl. 514/223.8

(57) ABSTRACT

Novel quaternary ammonium compounds of the formula

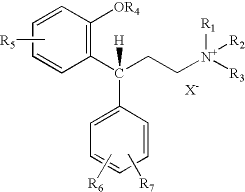

and any stereoisomers thereof, wherein R₁, R₂ R₃, R₄, R₅, R₆, R₇, and X⁻ are defined herein. The compounds are useful as medicaments for treatment of treatment of asthma, a group of breathing disorders termed Chronic Obstructive Pulmonary Disease (COPD), allergic rhinitis, rhinorrhea due to the common cold, and urinary disorder.

### CROSS REFERENCE TO RELATED APPLICATIONS

This application claims the benefit of the following provisional application: Application Ser. No. 60/462,956 filed Apr. 15, 2003 under 35 U.S.C. 119(e)(1).

### TECHNICAL FIELD

The present invention concerns a novel class of quaternary ammonium compounds, pharmaceutical compositions containing the same, the compounds for use as medicaments, and use of the compounds for the manufacture of specific medicaments. The present invention also concerns a method of treatment involving administration of the compounds.

The novel compounds are useful as antimuscarinic agents. In particular, the novel compounds are useful for the treatment of asthma, a group of breathing disorders termed Chronic Obstructive Pulmonary Disease (COPD), allergic rhinitis, rhinorrhea due to the common cold, and urinary disorder.

### BACKGROUND OF THE INVENTION

“Asthma” refers to a chronic lung disease causing bronchoconstriction (narrowing of the airways) due to inflammation (swelling) and tightening of the muscles around the airways. The inflammation also causes an increase in mucus production, which causes coughing that may continue for extended periods. Asthma is generally characterized by recurrent episodes of breathlessness, wheezing, coughing, and chest tightness, termed exacerbations. The severity of exacerbations can range from mild to life threatening. The exacerbations can be a result of exposure to e.g. respiratory infections, dust, mold, pollen, cold air, exercise, stress, tobacco smoke, and air pollutants.

“COPD” refers to Chronic Obstructive Pulmonary Disease, primarily associated with past and present cigarette smoking. It involves airflow obstruction, mainly associated with emphysema and chronic bronchitis. Emphysema causes irreversible lung damage by weakening and breaking the air sacs within the lungs. Chronic Bronchitis is an inflammatory disease, which increases mucus in the airways and bacterial infections in the bronchial tubes, resulting in obstructed airflow.

“Allergic rhinitis” refers to acute rhinitis or nasal rhinitis, including hay fever. It is caused by allergens such as pollen or dust. It may produce sneezing, congestion, runny nose, and itchiness in the nose, throat, eyes, and ears.

“Infectious rhinitis” refers to acute rhinitis or nasal rhinitis of infectious origin. It is caused by upper respiratory tract infection by infectious rhinoviruses, coronaviruses, influenza viruses, parainfluenza viruses, respiratory syncytical virus, adenoviruses, coxsackieviruses, echoviruses, or Group A beta-hemolytic Streptococci and generically referred to as the common cold. It may produce sneezing, congestion, runny nose, and itchiness in the nose, throat, eyes, and ears.

“Urinary disorders” and symptoms thereof include some or all of the following: urgency, frequency, incontinence, urine leakage, enuresis, dysuria, hesitancy, and difficulty of emptying bladder. In particular, urinary disorders include urinary incontinence, caused by e.g. unstable or overactive urinary bladder.

Overactive urinary bladder encompasses variants of urinary disorders, including overactive detrusor (detrusor instability, detrusor hyperreflexia) and sensory urgency, as well as symptoms of detrusor overactivity, e.g. urge incontinence, urgency, urinary frequency, and LUTS (Lower Urinary Tract Symptoms), including obstructive urinary symptoms, such as slow urination, dribbling at the end of urination, inability to urinate and/or the need to strain to urinate at an acceptable rate, or irritating symptoms such as frequency and/or urgency).

It is desirable to develop novel pharmaceutical compounds that further improve the quality of life for a large number of individuals.

### SUMMARY OF THE INVENTION

For these and other purposes, it is an object of the present invention to provide highly efficient pharmaceutical compounds for treatment of asthma.

It is also an object of the present invention to provide highly efficient pharmaceutical compounds for treatment of Chronic Obstructive Pulmonary Disease (COPD).

It is a further object of the present invention to provide highly efficient pharmaceutical compounds for treatment of allergic rhinitis.

It is an object of the present invention to provide highly efficient pharmaceutical compounds for treatment of rhinorrhea due to the common cold.

It is an object of the present invention to provide highly efficient pharmaceutical compounds for treatment of urinary disorder.

It is also an object of the present invention to provide pharmaceutically effective 3,3-diphenylpropylamine derivatives having an increased residence time in lung upon pulmonary administration.

It is another object of the invention to provide pharmaceutically effective 3,3-diphenylpropylamine derivatives with a prolonged systemic exposure and duration of action.

It is an object of the present invention to provide a novel class of 3,3-diphenylpropylamine derivatives having favorable properties.

For these and other objects that will be evident from the following disclosure, the present invention provides a quaternary ammonium compound of the formula

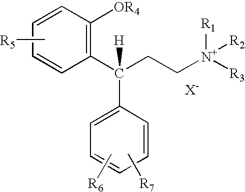

and any stereoisomers thereof, wherein

R₁, R₂ and R₃ are independently C₁-C₆-alkyl, C₃-C₇-cycloalkyl, C₃-C₆-alkenyl, C₄-C₈-cycloalkenyl, and C₃-C₆-alkynyl, wherein at least one of R₁, R₂ and R₃ contains an unsaturated carbon-carbon bond, and any two of R₁, R₂ and R₃ may form a ring together with the quaternary ammonium nitrogen, and the ring formed from any two of R₁, R₂ and R₃ may optionally contain an internal or exocyclic carbon-carbon double bond, and the ring formed from any two of R₁, R₂, and R₃ may additionally contain one or more substituents including C₁₋₄alkyl, C₂₋₄alkenyl, C₃₋₆alkynyl, aryl, halo, hydroxy, alkoxy, amino, and carboxyl.

R₄ is

—H,

—CH₃, or

—CO—R₄₋₁ wherein R₄₋₁ is

—(C₁-C₄ alkyl),

—(C₁-C₄ alkoxy), or

—NR₄₋₂R₄₋₃, wherein R₄₋₂ and R₄₋₃ are independently —H or —(C₁-C₄ alkyl),

and

R₅, R₆ and R₇ are independently

—H,

—OCH₃,

—OH,

—CONH₂,

—SO₂NH₂,

—F, —Cl, —Br, —I,

—CF₃, or

—(C₁-C₄ alkyl), optionally substituted with one or two

—OH,

—(C₁-C₄ alkoxy),

—COOH, or

—CO—O—(C₁-C₃ alkyl), and

X⁻ is an anion of a pharmaceutically acceptable acid.

In an embodiment of the compound according to the invention, the carbon stereocenter is (R). In another embodiment of the compound according to the invention, the carbon stereocenter is (S). In yet another embodiment, the compound according to the invention is a mixture of stereoisomers.

In one preferred embodiment of the compound according to the invention, R₁ and R₂ jointly form a ring together with the quaternary ammonium nitrogen. In a more preferred embodiment, said ring comprises from 4 to 6 carbon atoms.

In a preferred embodiment of the compound according to the invention, R₄ is —H, —CH₃, or —CO—R₄₋₁, wherein R₄₋₁ is C₁-C₄ alkyl. In a more preferred embodiment, R₄ is —H.

In a preferred embodiment of the compound according to the invention, R₅ is —H, —Br, —Cl, —CH₃, or —CH₂OH, more preferably —CH₃.

In a preferred embodiment of the compound according to the invention, at least one, more preferably both, of R₆ and R₇ is —H.

In a preferred embodiment of the compound according to the invention, X⁻ is selected from the group consisting of the anions of the following acids: tartaric, hydrochloric, hydrobromic, hydroiodic, sulfuric, phosphoric, nitric, citric, methanesulfonic, CH₃—(CH₂)ₙ—COOH where n is 0 thru 4, HOOC—(CH₂)n-COOH where n is 1 thru 4, HOOC—CH═CH—COOH and benzoic. In a more preferred embodiment, X⁻ is selected from the group consisting of iodide, bromide and chloride. In another preferred embodiment, X⁻ is iodide. In yet another preferred embodiment, X⁻ is chloride. In yet another preferred embodiment, X⁻ is bromide.

In another aspect the invention features a pharmaceutical composition including a therapeutically effective amount of a quaternary ammonium compound of formula I. The pharmaceutical composition may include a suitable pharmaceutical carrier.

In another aspect the present invention also provides a quaternary ammonium compound of formula I for use as a medicament. The present invention also includes using a quaternary ammonium compound of formula I for the manufacture of a medicament for treating asthma, urinary disorder, chronic obstructive pulmonary disease (COPD), allergic rhinitis, and infectious rhinitis.

In yet another aspect, the invention provides a method of treating asthma, chronic obstructive pulmonary disease (COPD), allergic rhinitis, or infectious rhinitis in a mammal, preferably a human, comprising administering to said mammal, in need of such a treatment, a therapeutically effective amount of a quaternary ammonium compound of formula I.

Finally, the present invention provides a method of treating asthma, chronic obstructive pulmonary disease (COPD), allergic rhinitis, rhinorrhea due to the common cold, or urinary disorder in a mammal, preferably a human, comprising administering to said mammal, in need of such a treatment, a therapeutically effective amount of a quaternary ammonium compound according to the invention.

### Definitions

The following definitions are used, unless otherwise described.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₇ alkyl refers to alkyl of one to seven carbon atoms, inclusive.

The term “halo” refers to a halogen atom selected from Cl, Br, I, and F.

The term “alkyl” refers to both straight- and branched-chain moieties. Unless otherwise specifically stated alkyl moieties include between 1 and 6 carbon atoms.

The term “alkenyl” refers to both straight- and branched-chain moieties containing at least one —C═C—. Unless otherwise specifically stated alkenyl moieties include between 1 and 6 carbon atoms.

The term “alkynyl” refers to both straight- and branched-chain moieties containing at least one —C≡C—. Unless otherwise specifically stated alkynyl moieties include between 1 and 6 carbon atoms.

The term “alkoxy” refers to —O-alkyl groups.

The term “cycloalkyl” refers to a cyclic alkyl moiety. Unless otherwise specifically stated cycloalkyl moieties will include between 3 and 7 carbon atoms.

The term “cycloalkenyl” refers to a cyclic alkenyl moiety. Unless otherwise specifically stated cycloalkenyl moieties will include between 3 and 7 carbon atoms and at least one —C═C— group within the cyclic ring.

The term “amino” refers to —NH₂.

The term “aryl” refers to phenyl and naphthyl.

The term “het” refers to mono- or bicyclic ring systems containing at least one heteroatom selected from O, S, and N. Each monocyclic ring may be aromatic, saturated, or partially unsaturated. A bicyclic ring system may include a monocyclic ring containing at least one heteroatom fused with a cycloalkyl or aryl group. A bicyclic ring system may also include a monocyclic ring containing at least one heteroatom fused with another het, monocyclic ring system. The term het encompasses the terms het¹, het², and heterocycloalkyl, described herein.

Examples of “het” include, but are not limited to, pyridine, thiophene, furan, pyrazoline, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl, 5-isothiazolyl, 1,3,4,-oxadiazole, 4-oxo-2-thiazolinyl, 5-methyl-1,3,4-thiadiazol-2-yl, thiazoledione, 1,2,3,4-thiatriazole, 1,2,4-dithiazolone, phthalimide, quinolinyl, morpholinyl, benzoxazoyl, diazinyl, triazinyl, quinolinyl, quinoxalinyl, naphthyridinyl, azetidinyl, pyrrolidinyl, hydantoinyl, oxathiolanyl, dioxolanyl, imidazolidinyl, and azabicyclo[2.2.1]heptyl.

The term “heteroaryl” refers to an aromatic het, examples of which include, but are not limited to, pyridine and thiophene.

### DESCRIPTION OF THE INVENTION

In describing the preferred embodiment, certain terminology will be utilized for the sake of clarity. Such terminology is intended to encompass the recited embodiments, as well as all technical equivalents that operate in a similar manner for a similar purpose to achieve a similar result. To the extent that any pharmaceutically active compound is disclosed or claimed, it is expressly intended to include all active metabolites produced in vivo, and, is expressly intended to include all enantiomers, isomers or tautomers where the compound is capable of being present in its enantiomeric, isomeric or tautomeric form.

The compounds of the invention can be prepared by one skilled in the art just by knowing the chemical structure of the compound to be prepared. The invention is the compounds themselves, not the process chemistry to make them. The chemistry is known to those skilled in the art.

For example, the compounds of the invention may be produced via the synthetic scheme shown in Chart I.

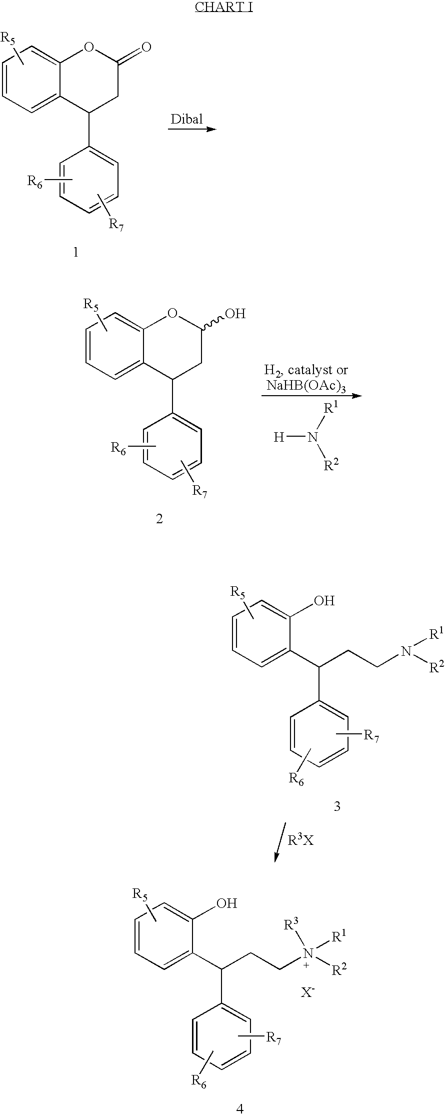

Referring to Chart 1, lactone 1 may be prepared by methods well known to those skilled in the art, e.g., by reacting an appropriately R5-substituted phenol with an appropriately R6-R7-substituted cinnamic acid under acidic or Lewis acidic conditions, resulting in lactone formation. Further methods of preparing lactone 1 may also be found in or adapted from, inter alia, Simpson, J. D. and Stephen, Henry., Journal of the Chemical Society, Abstracts (1956); Manimaran, T. and Ramakrishnan, V. T., Indian Journal of Chemistry, Section B: Organic Chemistry Including Medicinal Chemistry (1979), 18B(4), 324-30; Talapatra, Bani, Deb, Tulika and Talapatra, Sunil, Indian Journal of Chemistry, Section B: Organic Chemistry Including Medicinal Chemistry (1986), 25B(11), 1122-5; Bhattacharjee, J. and Paknikar, S. K., Indian Journal of Chemistry, Section B: Organic Chemistry Including Medicinal Chemistry (1989), 28B(3), 205-7; and Kirtany, J. K., Indian Journal of Chemistry, Section B: Organic Chemistry Including Medicinal Chemistry (1993), 32B(9), 993. It will be appreciated by those skilled in the art that the corresponding appropriately-substituted phenols and cinnamic acids may be prepared by methods well known to those skilled in the art.

Reduction of the lactone 1 using diisobutylaluminum hydride (Dibal) provides the lactol 2. Reductive amination of the lactol with a catalyst, such as palladium in the presence of hydrogen, or with NaHB(OAc)₃, and a secondary amine provides the tertiary amine 3. Reacting the tertiary amines with the desired allyl, alkyl, or benzyl halide provides the desired quartemary ammonium compounds.

Although shown as the phenol, the hydroxy group may be derivatized prior to quarternization. For instance, reaction of the phenol hydroxy group with an acid chloride or with an acid and a coupling agent produces esters which may be further derivatized such as with an isocyanate to produce urethanes.

Accordingly, the compounds of the present invention are quaternary ammonium compounds and are prepared by means, well known to those skilled in the art, for preparing quaternary ammonium compounds from tertiary amines, using the tertiary amines of U.S. Pat. No. 5,382,600 and other known compounds as starting materials. The general term “quaternary ammonium compound” relates to any compound that can be regarded as derived from ammonium hydroxide or an ammonium salt by replacement of all four hydrogen atoms of the NH₄⁺-ion by organic groups.

The specific compounds are for nomenclature reasons (see e.g. Chemical Abstracts) named as “aminium” compounds, but it is possible to use the term “ammonium” in the names. For example, (3R)-3-(2-hydroxy-5-methylphenyl)-N,N-diisopropyl-N-methyl-3-phenylpropan-1-aminium bromide can also be named as an ammonium compound: (3R)-[3-(2-hydroxy-5-methylphenyl)-3 -phenylpropyl]diisopropylmethylammonium bromide.

More specifically, the invention concerns quaternary ammonium compounds of the formula:

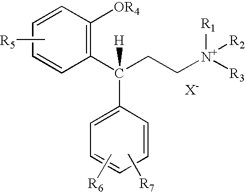

and any stereoisomers thereof, wherein

R₁, R₂ and R₃ independently represent C₁-C₆-alkyl, C₃-C₇-cycloalkyl, C₃-C₆-alkenyl, C₄-C₈-cycloalkenyl, and C₃-C₆-alkynyl, wherein at least one of R₁, R₂ and R₃ contains an unsaturated carbon-carbon bond, and any two of R₁, R₂ and R₃ may form a ring together with the quaternary ammonium nitrogen, and the ring formed from any two of R₁, R₂ and R₃ may optionally contain an internal or exocyclic carbon-carbon double bond, and the ring formed from any two of R₁, R₂, and R₃ may additionally contain one or more substituents including C₁₋₄alkyl, C₂₋₄alkenyl, C₃₋₆alkynyl, aryl, halo, hydroxy, alkoxy, amino, and carboxyl.

R₄ represents

—H,

—CH₃, or

—CO—R₄₋₁ wherein R₄₋₁ represents

—(C₁-C₄ alkyl),

—(C₁-C₄ alkoxy), or

—NR₄₋₂R₄₋₃, wherein R₄₋₂ and R₄₋₃ independently represent —H or —(C₁-C₄ alkyl),

and

R₅, R₆ and R₇ independently represent

—H,

—OCH₃,

—OH,

—CONH₂,

—SO₂NH₂,

—F, —Cl, —Br, —I,

—CF₃, or

—(C₁-C₄ alkyl), optionally substituted with one or two

—OH,

—(C₁-C₄ alkoxy),

—COOH, or

—CO—O—(C₁-C₃ alkyl), and

X⁻ represents an anion of a pharmaceutically acceptable acid.

By way of example, a tertiary amine according to U.S. Pat. No. 5,382,600, or its salt, is dissolved in a suitable solvent. The tertiary amine is allowed to react with an organic substrate, e.g. an organic halide.

The substrate contains a C₃-C₇ alkenyl, preferably a C₃-C₅ alkenyl and a leaving group. The identity of the leaving group is not critical, but it is preferred that the leaving group is a halide, such as iodide or bromide. Thus, exemplary substrates include allyl bromide, allyl iodide, 2-methylprop-2-enyl bromide, 2-methylprop-2-enyl iodide, cis-1-bromo-2-butene, cis-1-iodo-2-butene, trans-1-bromo-2-butene, trans-1-iodo-2-butene, 1-bromo-3-methyl-2-butene, or 1-iodo-3-methyl-2-butene.

The resulting reaction product is a quaternary ammonium compound, which is readily crystallized in suitable solvents, known to those skilled in the art. The crystals thus produced are quaternary ammonium salts. Their identity is confirmed by standard methods, such as melting point determination, nuclear magnetic resonance (NMR) analysis and mass spectrometry.

The quaternary ammonium compounds of the invention have at least one stereocenter, i.e. the carbon in position 3 (C₃ in the formula below), to which two (substituted) aryl groups are attached. Optionally, there may be a second stereocenter (when R₁, R₂ and R₃ all are different), the positively charged quaternary ammonium nitrogen atom. See the general formula:

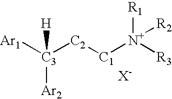

wherein Ar₁ and Ar₂ denote (substituted) aryl groups, R₁, R₂, R₃ and X⁻ are as above, and C₁, C₂ and C₃ denote individual carbon atoms in the propylammonium backbone. Accordingly, stereoisomers (enantiomers and/or diastereomers) are produced. All stereoisomers have useful activity. Therefore, the invention includes use of each stereoisomer separately, as well as mixtures thereof. Specifically, the stereoisomers in which the C₃ carbon stereocenter is in the (R) form have useful activity. Moreover, the stereoisomers in which the C₃ carbon stereocenter is in the (S) form have useful activity. A mixture of stereoisomers, comprising the stereoisomers in which the C₃ carbon stereocenter is in the (R) form and the stereoisomers in which the C₃ carbon stereocenter is in the (S) form, also has useful activity.

The quaternary ammonium compounds of the invention are preferably administered as salts with a pharmaceutically acceptable acid. Where R₄ is —H, the compounds can be isolated as internal salts, which have a phenoxide anion to balance the positive charge on the quaternized nitrogen. The preferred pharmaceutically acceptable salts include salts of the following acids: tartaric, hydrochloric, hydrobromic, hydroiodic, sulfuric, phosphoric, nitric, citric, methanesulfonic, CH₃—(CH₂)ₙ—COOH where n is 0 thru 4, HOOC—(CH₂)ₙ—COOH where n is 1 thru 4, HOOC—CH═CH—COOH, and benzoic. For other acceptable salts, see Int. J. Pharm., 33, 201-217 (1986). Particularly preferred salts are chloride, iodide and bromide salts, especially bromide salts and iodide salts.

Accordingly, X⁻ represents an anion of a pharmaceutically acceptable acid. Preferably, X⁻ is selected from the following anions: tartrate, chloride, bromide, iodide, sulfate, hydrogen sulfate, phosphate(s), hydrogen phosphate(s), nitrate, citrate, methanesulfonate, carboxylates with from two to six carbon atoms, dicarboxylates with from two to six carbon atoms, maleate, fumarate, and benzoate. Specifically X⁻ may represent chloride, iodide or bromide.

In certain embodiments, the substituents R₁, R₂, R₃ may be the same or different. They are selected from the group including C₂-C₆ alkenyls, preferably C₂-C₅ alkenyls, straight or branched. At least one of the substituents R₁, R₂, R₃ represents a C₂-C₄ alkenyl, straight or branched, such as allyl (prop-2-enyl).

According to another aspect of the invention, any two of R₁, R₂, and R₃ may jointly form a ring structure together with the positively charged nitrogen. It is preferred that the resulting ring structure comprises from four to six carbon atoms.

The substituent R₄ is attached via an oxygen atom to its aryl ring. The —OR₄ group is attached to the carbon atom in position 2 in the ring, with respect to the propylammonium group. The substituent R₄ may represent hydrogen, methyl or acyl (—CO—R₄₋₁), wherein acyl includes any one of the following: alkylcarbonyl, straight or branched, having from two to five carbon atoms, alkoxycarbonyl, straight or branched, having from two to five carbon atoms, and amide, optionally mono- or independently disubstituted with alkyl, straight or branched, having from one to four carbon atom(s). Accordingly, the substituent R₄₋₁, represents any one of the following: C₁-C₄ alkyl, straight or branched, C₁-C₄ alkoxy, straight or branched, and —NR₄₋₂R₄₋₃, wherein R₄₋₂ and R₄₋₃ may be the same or different and represent —H or —(C₁-C₄ alkyl), straight or branched. Thus, the substituent R₄ may represent any one of the following: hydrogen, methyl or acyl, wherein the acyl group may be acetyl (ethanoyl), propanoyl, butanoyl, isobutanoyl, pentanoyl, isopentanoyl, methoxycarbonyl, ethoxycarbonyl, propoxycarbonyl, isopropoxycarbonyl, butoxycarbonyl, isobutoxycarbonyl, carbamoyl, N-methylcarbamoyl, N-ethylcarbamoyl, N-propylcarbamoyl, N-butylcarbamoyl, or an N,N-dialkylcarbamoyl, wherein the alkyl groups, straight or branched, are the same or different and have from 1 to 4 carbon atoms each. Examples of N,N-dialkylcarbamoyls in this position include N,N-dimethylcarbamoyl, N,N-diethylcarbamoyl, N,N-dipropylcarbamoyl, as well as N,N-diisobutylcarbamoyl, and N-propyl-N-butylcarbamoyl. It is preferred that R₄ represents hydrogen, since such compounds can be isolated as internal salts, which have a phenoxide anion to balance the positive charge on the quaternized nitrogen. It is also preferred that R₄ represents alkylcarbonyl, straight or branched, having from two to five carbon atoms, e.g. acetyl (ethanoyl), propanoyl, butanoyl, isobutanoyl, pentanoyl, or isopentanoyl. Moreover, it is preferred that R₄ represents methyl.

The substituent R₅ may be connected to any, otherwise not substituted, carbon atom in its aryl ring. In other words, R₅ is not connected to any of the carbon atoms to which the —OR₄ group or the (substituted) phenylpropanammonium group is connected, but R₅ may be connected to any one of the remaining four carbon atoms in its aryl ring.

R₅ may represent any one of the following: hydrogen, methoxy, hydroxyl, carbamoyl, sulphamoyl, halogen (fluorine, chlorine, bromine, iodine), trifluoromethyl or an alkyl group, straight or branched, having from one to four carbon atoms. Optionally, this alkyl group may be mono- or independently disubstituted with hydroxyl, with an alkoxy group, straight or branched, having from one to four carbon atoms, with carboxyl, or with alkoxycarbonyl (—CO—O—(C₁-C₃ alkyl)), straight or branched, having from one to four carbon atoms. It is preferred that R₅ represents any one of the following: hydrogen, bromine, chlorine, methyl or hydroxymethyl. It is particularly preferred that R₅ represents methyl. If R₅ does not represent hydrogen, it is preferred that R₅ is situated opposite the —OR₄ group, i.e. at the carbon atom in position 5 in the ring, with respect to the propylammonium group.

The substituents R₆ and R₇ are connected to the same aryl ring, which is different from the aryl ring to which the substituents R₄ and R₅ are connected. R₆ and R₇ may be the same or different. R₆ and R₇ may independently represent any one of the following: hydrogen, methoxy, hydroxyl, carbamoyl, sulphamoyl, halogen (fluorine, chlorine, bromine, iodine), trifluoromethyl or an alkyl group, straight or branched, having from one to four carbon atoms. Optionally, this alkyl group may be mono- or independently disubstituted with hydroxyl, with an alkoxy group, straight or branched, having from one to four carbon atoms, with carboxyl, or with alkoxycarbonyl (—CO—O—(C₁-C₃ alkyl)), straight or branched, having from one to four carbon atoms.

It is preferred that at least one, preferably both, of R₆ and R₇ represents hydrogen. When one, but not both, of R₆ and R₇ represents hydrogen, it is preferred that the other (R₇ or R₆, respectively) is attached to the carbon atom in position 2 in the ring, with respect to the propylammonium group. When neither R₆ nor R₇ represent hydrogen, it is preferred that one is attached to the carbon atom in position 2 and the other to any one of the carbon atoms in positions 3, 4, or 5, respectively, in the ring, with respect to the propylammonium group.

The novel class of compounds according to the present invention are antimuscarinic agents. “Antimuscarinic agents” refer to muscarinic receptor antagonists. Examples of known antimuscarinic agents include tolterodine, hydroxytolterodine, 2-(diisopropylamino)ethyl-1-phenylcyclopentanecarboxylate, propiverine, oxybutynin, trospium, darifenacin, temiverine, ipratropium, and tiotropium.

Propiverine is 1-methyl-4-piperidyl-α, α-diphenyl-α-(n-propoxy)acetate and is disclosed in East German Patent 106,643 and in CAS 82-155841s (1975). Oxybutynin is 4-(diethylamino)-2-butynylalphaphenyl cyclohexaneglycolate and is disclosed in UK Patent 940,540. Trospium is 3-α-hydroxyspiro[1-α-H, 5-α-H-nortropane-8,1′-pyrrolidinium]chloride benzilate and is disclosed in U.S. Pat. No. 3,480,623. Darifenacin is 3-Pyrrolidineacetamide, 1-[2-(2,3-dihydro-5-benzofuranyl)ethyl]-α,α-diphenyl-, and is disclosed in U.S. Pat. No. 5,096,890. Temiverine is benzeneacetic acid, α-cyclohexyl-α-hydroxy-, 4-(diethylamino)-1,1-dimethyl-2-butynyl ester and is disclosed in U.S. Pat. No. 5,036,098. Ipratropium is 8-isopropylnoratropine methobromide and is disclosed in U.S. Pat. No. 3,505,337. Tiotropium is (1-α, 2-β, 4-β, 5-α, 7-β)-7-[(hydroxydi-(2-thienyl)acetyl)oxy]-9,9-dimethyl-3-oxa-9-azoniatricyclo[3.3.1.02,4]nonaneand is disclosed in EP 418,716.

The compounds of the invention have anti-cholinergic properties. Thus, they are useful for the treatment of acetylcholine-mediated disorders. In particular, they are useful for treating asthma, chronic obstructive pulmonary disease (COPD), allergic rhinitis, rhinorrhea due to the common cold, and urinary disorder.

Other conditions are also included, which give rise to urinary frequency, urgency and/or urge incontinence. Overactive bladder disorders also include nocturia and mixed incontinence. While overactive bladder is often associated with detrusor muscle instability, disorders of bladder finction may also be due to neuropathy of the central nervous system (detrusor hyperreflexia), including spinal cord and brain lesions, such as multiple sclerosis and stroke. Overactive bladder symptoms may also result from, for example, male bladder outlet obstruction (usually due to prostatic hypertrophy), interstitial cystitis, local edema and irritation due to focal bladder cancer, radiation cystitis due to radiotherapy to the pelvis, and cystitis.

A specific problem which can be treated by the claimed method is a dry overactive bladder, which includes frequency, urgency and nocturia.

The compounds of the present invention are used to treat mammals, including man and horse. It is preferred that the mammal is a human.

The compounds according to the invention, in the form of free base or salts with pharmaceutically acceptable acids, or solutions thereof, can be brought into suitable dosage forms, such as compositions for administration through the oral, rectal, transdermal, parenteral, nasal, or pulmonary route in accordance with accepted pharmaceutical procedures. In particular, the compositions may be administered via inhalation or insufflation. Such pharmaceutical compositions according to the invention comprise the compounds according to the invention in association with compatible pharmaceutically acceptable carrier materials, or diluents, as is well known in the art. The carriers may be any inert material, organic or inorganic, suitable for administration, such as: water, gelatin, gum arabicum, lactose, microcrystalline cellulose, starch, sodium starch glycolate, calcium hydrogen phosphate, magnesium stearate, talcum, colloidal silicon dioxide, and the like. Such compositions may also contain other pharmaceutically active agents, and conventional additives such as stabilizers, wetting agents, emulsifiers, flavoring agents, buffers, binders, disintegrants, lubricants, glidants, antiadherents, propellants, and the like.

The novel compounds according to the present invention can be administered in any suitable way. The compounds according to the invention can be made up in solid or liquid form, such as tablets, capsules, powders, syrups, elixirs and the like, aerosols, sterile solutions, suspensions or emulsions, and the like. They are advantageously administered via inhalation or insufflation. When the administration form is inhalation or insufflation, the compounds are preferably in the form of either an aerosol or a powder.

The term “effective amount” refers to a therapeutically effective amount for treating asthma, chronic obstructive pulmonary disease (COPD), allergic rhinitis, rhinorrhea due to the common cold, or urinary disorder. The terms “therapy” and “therapeutically” encompass all kinds of treatments, including prophylaxis. In particular, “therapeutically effective” means that it is effective for anti-cholinergic treatment.

The dosage of the specific compound according to the invention will vary depending on its potency, the mode of administration, the age and weight of the patient and the severity of the condition to be treated.

Doses administrated by inhaler, such as a dry powder inhaler (DPI) or a metered-dose inhaler (MDI), are preferably given as one or two puffs, preferably comprising the total daily dose. For a human subject, it is preferred that the dosage is in the range of from 1 microgram (1 μg) to one milligram (1 mg).

Doses administrated by nebulizer solution are generally higher than doses administrated by inhaler. For a human subject, it is preferred that the total dosage given by nebulizer solution is in the range of from 1 microgram (1 μg) to ten milligrams (10 mg).

Thus, a clinically effective amount of the compounds according to the invention is from about 1 μg to about 10 mg. It is preferred that the effective amount is from about 1 μg to about 1 mg, preferably from about 0.01 mg to about 1 mg.

The compounds of the invention can be administered from one to four times daily. It is preferable to administer the compounds once or twice daily, more preferable once daily.

The dosage form for inhalation can be an aerosol. The minimum amount of an aerosol delivery is about 0.2 ml and the maximum aerosol delivery is about 5 ml. The concentration of the compounds according to the invention may vary as long as the total amount of spray delivered is within the about 0.2 to about 5 ml amount and it delivers an effective amount. It is well known to those skilled in the art that if the concentration is higher, one gives a smaller dose to deliver the same effective amount.

The dosage form for inhalation can also be via intranasal spray. The minimum amount of an aerosol delivery is about 0.02 ml per nostril and the maximum aerosol delivery is about 0.2 ml per nostril. The concentration of the compounds according to the invention may vary as long as the total amount of spray delivered is within about 0.02 ml per nostril to about 0.2 ml per nostril, e.g., between about 0.05 ml per nostril and about 0.08 ml per nostril, and it delivers a therapeutically effective amount of the compound of formula I.

Of course, the volume of aerosol or intranasal spray for delivering a therapeutically effective amount of the compound of formula I depends upon the concentration of the compound in the aerosol or intranasal spray, e.g., higher concentrations of the compound of formula I require smaller dosage volumes to deliver a therapeutically effective amount and lower concentrations of the compound of formula I require larger dosage volumes to deliver the same therapeutically effective amount.

Aerosols for inhalation of various pharmaceutical agents are well known to those skilled in the art, including many aerosols for treating asthma. Aerosols may be produced with a nebulizer. Typically, the nebulizer is charged with a carrier solution and the compound of formula I in an amount sufficient to effectively deliver a therapeutically effective amount of the antimuscarininc compound. For instance, depending upon the nebulizer and its operating conditions, the nebulizer may be charged with several hundred mg of antimuscarinic compound in order to deliver about 1 μg to about 1000 μg, e.g., from about 10 μg to about 1000 μg or from about 50 μg to about 500 μg, of the compound of formula I.

The non-active ingredient or carrier can be just (sterile) water with the pH adjusted to where the active pharmaceutical agent is very soluble. It is preferred that the pH be at or near 7. Alternatively and preferably, the non-active carrier agent should be physiological saline with the pH adjusted appropriately. Aerosols for inhalation of various pharmaceutical agents are well known to those skilled in the art, including many aerosols for treating asthma.

Alternatively, the dosage form for inhalation can be a powder. Powders for inhalation of various pharmaceutical agents are well known to those skilled in the art, including many powders for treating asthma. When the dosage form is a powder, the compounds according to the invention can be administered in pure form or diluted with an inert carrier. When an inert carrier is used, the compounds according to the invention are compounded such that the total amount of powder delivered delivers an “effective amount” of the compounds according to the invention. The actual concentration of the active compound may vary. If the concentration is lower, then more powder must be delivered; if the concentration is higher, less total material must be delivered to provide an effective amount of the active compound according to the invention.

For treatment of rhinitis, in particular rhinitis due to the common cold, the compounds according to the invention can advantageously be administered in combination with steroids, cromoglycates, and decongestants (alpha agonists). Such combination therapies are useful in the treatment of rhinorrhea due to the common cold.

The invention will be further illustrated by the following non-limiting examples and pharmacological tests.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

All temperatures are in degrees Celsius.

Ether refers to diethyl ether.

Physiological saline refers to an 0.9% aqueous sodium chloride solution.

When solvent pairs are used, the ratios of solvents used are volume/volume (v/v).

When the solubility of a solid in a solvent is used the ratio of the solid to the solvent is weight/volume (wt/v).

Reductive Amination

General Procedure A:

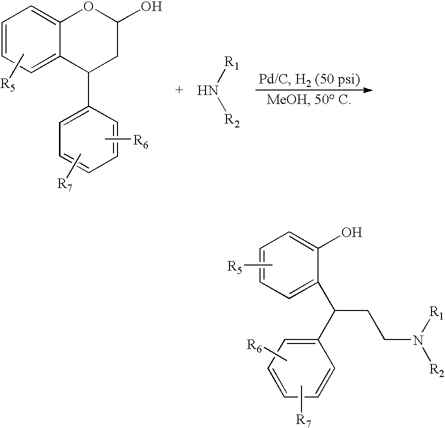

Palladium on activated carbon (1.76 g, 5% wt, Aldrich 20,568-0) was charged to a hydrogenation vessel under nitrogen followed by a MeOH (20 mL) solution of racemic lactol (4 g, 16.64 mmol) and a secondary amine (42 mmol, 2.5 equiv.). The vessel was filled with hydrogen (50 psi) and the reaction mixture was stirred vigorously at 50° C. overnight. The heterogeneous reaction mixture was filtered through celite. The resulting methanolic solution was concentrated under vacuum.

Cyclic amines, where R₁ and R₂ and the nitrogen form a ring, were obtained after trituration with hexanes.

General Procedure B:

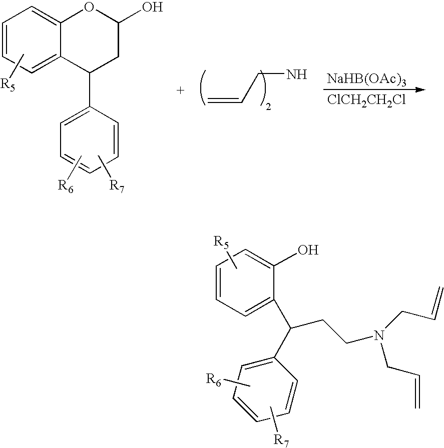

Solid NaHB(OAc)₃ (3 g, 14 mmol) was added under nitrogen to a solution of racemic lactol (2.4 g, 10 mmol) and secondary amine (0.97 g, 1.23 mL, 10 mmol) in 1,2-dichloroethane (35 mL). The reaction mixture was stirred overnight at room temperature. The reaction mixture was quenched with a saturated aqueous solution of NaHCO₃, layers were separated and the aqueous layer was extracted with ether (2×30 mL). The combined organic layers were dried over MgSO₄. After filtration, the solvents were removed under vacuum to give the crude tertiary amine as an oil. The tertiary amine obtained following this procedure was used without purification for the quaternization step.

Quaternization of the Tertiary Amines

General Procedure

Alkyl, benzyl, or allyl including a counter anion such as halide (10 equivalents) were added to a solution of free base of the tertiary amine (0.3 g, 1.02 mmol) in acetone (4 mL). The reaction mixture is stirred overnight at room temperature. The solution is concentrated to initiate the precipitation of the quaternary ammonium salt. The white precipitate is filtered, washed with diethyl ether and dried under vacuum to give the corresponding quatemized salts.

### Example 1

### 1-[3-(2-Hydroxy-5-methylphenyl)-3-phenylpropyl-1-(2-methylprop-2-enyl)pyrrolidinium Bromide

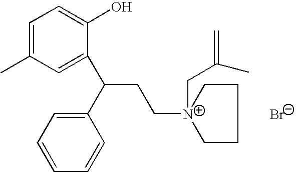

The title compound was produced by reductive amination of 6-methyl-4-phenyl-2-chromanol (Compound 2 from Chart 1) with pyrrolidine followed by quatemization with prop-2-enyl bromide according to the procedures described above. ¹H NMR (MeOH-d₄): δ 1.90, 2.0-2.25, 2.47-2.71, 3.21-3.31, 3.50-3.64, 3.97, 4.38, 5.36, 5.41, 6.70, 6.88, 6.95, 7.18-7.24, 7.25-7.40.

### Example 2

### 1-[3-(2-Hydroxy-5-methylphenyl)-3-phenylpropyl]-1-(3-methylbut-2-enyl)pyrrolidinium Bromide

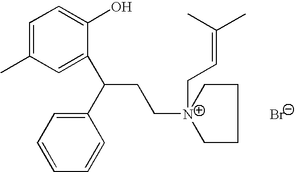

The title compound was produced by reductive amination of 6-methyl-4-phenyl-2-chromanol with pyrrolidine followed by quatemization with 3-methylbut-2-enyl bromide according to the procedures described above. ¹H NMR (MeOH-d₄): δ 1.88, 1.90, 2.0-2.25, 2.40-2.65, 3.18-3.24, 3.38-3.60, 3.97, 4.38, 5.20, 5.41, 6.68, 6.88, 6.95, 7.18-7.24, 7.25-7.40.

### Example 3

### 1-Allyl-1-[3-(2-hydroxy-5-methylphenyl)-3-phenylpropyl]pyrrolidinium Iodide

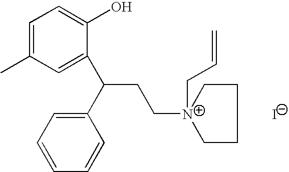

The title compound was produced by reductive amination of 6-methyl-4-phenyl-2-chromanol with pyrrolidine followed by quaternization with allyl iodide according to the procedures described above. ¹H NMR (MeOH-d₄): δ 2.0-2.25, 2.40-2.70, 3.17-3.29, 3.38-3.61, 3.97, 4.38, 5.26-5.70, 5.80-6.01, 6.68, 6.88, 6.97, 7.18-7.24, 7.25-7.40.

### Example 4

### 1-Allyl-1-[3-(2-hydroxy-5-methylphenyl)-3-phenylpropyl]pyrrolidinium Chloride

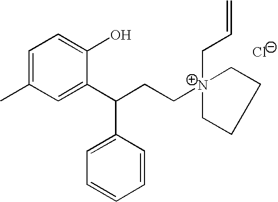

The title compound was produced via an ion-exchange reaction. The iodide compound of Example 3 (0.6 g) was vigorously stirred with the chloride form of ion-exchange resin AG-2-X8 Bio-Rad (60 g) in 200 mL of an acetonitrile/water mixture (30/70) for 4h. The resin was filtered on a sintered glass funnel and washed with an acetonitrile/water mixture (30/70) (40 ml). The acetonitrile was removed under vacuum and the remaining water was removed on a lyophilizer to give 0.35 g (72%) of a slightly off-white solid of the titled compound. ¹H NMR (MeOH-d₄): δ 2.0-2.25, 2.40-2.70, 3.17-3.29, 3.38-3.61, 3.97, 4.38, 5.26-5.70, 5.80-6.01, 6.68, 6.88, 6.97, 7.18-7.24, 7.25-7.40.

### Example 5

### 3-(2-Hydroxy-5-methylphenyl)-N,N-diallyl-N-methyl-3-phenyl propan-1-aminium Iodide

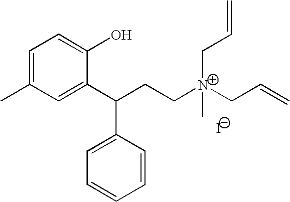

### Preparation of 2-[3-(diallylamino)-1-phenylpropyl]-4-methylphenol

The tertiary amine was propuced by reductive amination of the lactol according to the procedures described above.

### Preparation of 3-(2-Hydroxy-5-methylphenyl)-N,N-diallyl-N-methyl-3-phenyl propan-1-aminium Iodide

Methyl iodide (2.2 g, 0.96 mL, 0.0155 mol) was added to a solution of the tertiary amine (0.5 g, 1.55 mmol) in a mixture of ether (3 mL) and acetone (1 mL). The reaction mixture was stirred overnight at room temperature to give a white precipitate. The white precipitate was filtered out, triturated with ether, filtered and dried under vacuum to give the title compound. ¹H NMR (MeOH-d₄): δ 2.19, 2.48-2.67, 2.98, 3.1-3.28, 3.96, 4.36, 5.61-5.7, 5.86-6.00, 6.68, 6.84, 7.01, 7.18, 7.29, 7.38.

### Example 6

### 3-(2-Hydroxy-5-methylphenyl)-N,N-diallyl-N-ethyl-3-phenylpropan-1-aminium Iodide

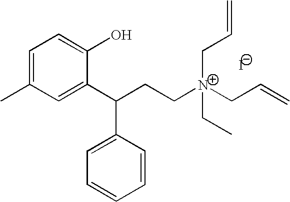

Ethyl iodide (2.42 g, 1.24 mL, 0.0155 mol) was added to a solution of 2-[3-(diallylamino)-1-phenylpropyl]-4-methylphenol (0.5 g, 1.55 mmol) in acetone (3 mL). The reaction mixture was stirred overnight at room temperature to give a white precipitate. The white precipitate was filtered then washed with ether and dried under vacuum to give the title compound. ¹H NMR (MeOH-d₄): δ 1.25, 2.19, 2.44-2.65, 3.09-3.22, 3.29-3.36, 3.91, 4.35, 5.6-5.7, 5.85-5.99, 6.8, 6.85, 7.0, 7.19, 7.30, 7.39.

### Example 7

### 1-Allyl-1-[3-(2-hydroxy-5-methylphenyl)-3-phenyl propyl]piperidinium Chloride

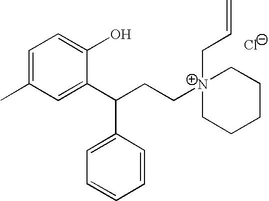

1-[3-(2-hydroxy-5-methylphenyl)-3-phenyl propyl]piperidine was prepared by reductive amination of the lactol with piperidine according to the procedures described above.

Allyl iodide (1.64 g, 0.88 mL, 0.098 mol) was added to a solution of 1-[3-(2-hydroxy-5-methylphenyl)-3-phenyl propyl]piperidine (0.3 g, 0.97 mmol) in a mixture of acetonitrile (6 mL) and methylene chloride (3 mL). The reaction mixture was stirred overnight at room temperature. The solvents were removed under vacuum and the resulting solid triturated with ether to give a solid. The solid was vigorously stirred with the chloride form of ion-exchange resin AG-2-X8 (70 g) in 200 mL of an acetonitrile/water mixture (30/70) for 4 h. The acetonitrile was removed under vacuum and the remaining water removed on a lyophilizer to give the title compound. ¹H NMR (MeOH-d₄): δ 1.64-1.83, 2.19, 2.4-2.59, 3.15-3.33, 4.0, 4.36, 5.56-5.66, 5.76-587, 6.68, 6.85, 7.19, 7.28-7.39.

### Example 8

### 3-(2-Hydroxy-5-methylphenyl)-N,N,N-triallyl-3-phenylpropan-1-aminium Bromide

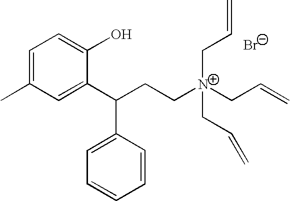

Allyl bromide (1.88 g, 1.34 mL, 0.0155 mol) was added to a solution of 2-[3-(diallylamino)-1-phenylpropyl]-4-methylphenol (0.5 g, 1.55 mmol) in acetone (3 mL). The reaction mixture was stirred overnight at room temperature to give a white precipitate. The white precipitate was filtered out, washed with ether and dried under vacuum to give the title compound. ¹H NMR (MeOH-d₄): δ 2.18, 2.47-2.67, 3.09-3.26, 3.92, 4.34, 5.64-5.70, 5.9-6.04, 6.68, 6.85, 6.92, 7.20, 7.28-7.37.

We claim:

1. A quaternary ammonium compound of the formula 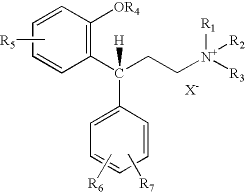
or a stereoisomer thereof, wherein
R₁, R₂ and R₃ are independently C₁-C₆-alkyl, C₃-C₇-cycloalkyl, C₃-C₆-alkenyl, C₄-C₈-cycloalkenyl, and C₃-C₆-alkynyl, wherein at least one of R₁, R₂ and R₃ contains an unsaturated carbon-carbon bond, and any two of R₁, R₂ and R₃ may form a ring together with the quaternary ammonium nitrogen, and the ring formed from any two of R₁, R₂ and R₃ may optionally contain an internal or exocyclic carbon-carbon double bond, and the ring formed from any two of R₁, R₂, and R₃ may additionally be substituted with one to three C₁₋₄alkyl, C₂₋₄alkenyl, C₃₋₆alkynyl, aryl, halo, hydroxy, alkoxy, amino or carboxyl;
R₄ is
—H,
—CH₃, or
—CO—R₄₋₁ wherein R₄₋₁ is
—(C₁-C₄ alkyl),
—(C₁-C₄ alkoxy), or
—NR₄₋₂R₄₋₃, wherein R₄₋₂ and R₄₋₃ are independently —H or —(C₁-C₄ alkyl);
and
R₅, R₆ and R₇ are independently
—H,
—OCH₃,
—OH,
—CONH₂,
—SO₂NH₂,
—F, —Cl, —Br, —I,
—CF₃, or
—(C₁-C₄ alkyl), optionally substituted with one or two
—OH,
—(C₁-C₄ alkoxy),
—COOH, or
—CO—O—(C₁-C₃ alkyl), and
X⁻ is an anion of a pharmaceutically acceptable acid.

2. A quaternary ammonium compound according to claim 1, wherein the carbon stereocenter is (R).

3. A quaternary ammonium compound according to claim 1, wherein the carbon stereocenter is (S).

4. A quaternary ammonium compound according to claim 1, which is a mixture of stereoisomers.

5. A quaternary ammonium compound according to claim 1, wherein at least one of R₁, R₂ and R₃ is C₂-C₅ alkenyl.

6. A quaternary ammonium compound according to claim 5, wherein at least one of R₁, R₂ and R₃ is allyl.

7. A quaternary ammonium compound according to claim 6, wherein at least two of R₁, R₂ and R₃ is allyl.

8. A quaternary ammonium compound according to claim 5, wherein at least one of R₁, R₂ and R₃ is methyl.

9. A quaternary ammonium compound according to claim 5, wherein at least one of R₁, R₂ and R₃ is ethyl.

10. A quaternary ammonium compound according to claim 1, wherein R₁ and R₂ jointly form a ring together with the quaternary ammonium nitrogen.

11. A quaternary ammonium compound according to claim 10, wherein said ring comprises from 4 to 8 carbon atoms.

12. A quaternary ammonium compound according to claim 1, wherein R₄ is —H, —CH₃, or —CO—R₄₋₁, wherein R₄₋₁ is C₁-C₄ alkyl.

13. A quaternary ammonium compound according to claim 12, wherein R₄ is —H.

14. A quaternary ammonium compound according to claim 1, wherein R₅ is —H, —Br, —Cl, —CH₃, or —CH₂OH.

15. A quaternary ammonium compound according to claim 14, wherein R₅ is —CH₃.

16. A quaternary ammonium compound according to claim 1, wherein at least one of R₆ and R₇is —H.

17. A quaternary ammonium compound according to claim 1, wherein both R₆ and R₇ represent —H.

18. A quaternary ammonium compound according to claim 1, wherein X⁻ is selected from the group consisting of the anions of the following acids: tartaric, hydrochloric, hydrobromic, hydroiodic, sulfuric, phosphoric, nitric, citric, methanesulfonic, CH₃—(CH₂)ₙ—COOH where n is 0 thru 4, HOOC—(CH₂)n-COOH where n is 1 thru 4, HOOC—CH═CH—COOH and benzoic.

19. A quaternary ammonium compound according to claim 18, wherein X⁻ is selected from the group consisting of iodide, bromide and chloride.

20. A quaternary ammonium compound according to claim 19, wherein X⁻ is iodide.

21. A quaternary ammonium compound according to claim 19, wherein X⁻ is chloride.

22. A quaternary ammonium compound according to claim 19, wherein X⁻ is bromide.

23. 1-[3-(2-Hydroxy-5-methylphenyl)-3-phenylpropyl]-1-(2-methylprop-2-enyl)pyrrolidinium bromide;
1-[3-(2-hydroxy-5-methylphenyl)-3-phenylpropyl]-1-(3-methylbut-2-enyl)pyrrolidinium bromide;
1-allyl-1-[3-(2-hydroxy-5-methylphenyl)-3-phenylpropyl]pyrrolidinium iodide;
1-allyl-1-[3-(2-hydroxy-5-methylphenyl)-3-phenylpropyl]pyrrolidinium chloride;
3-(2-hydroxy-5-methylphenyl)-N,N-diallyl-N-methyl-3-phenyl propan-1-aminium iodide;
3-(2-hydroxy-5-methylphenyl)-N,N-diallyl-N-ethyl-3-phenylpropan-1-aminium iodide;
1-allyl-1-[3-(2-hydroxy-5-methylphenyl)-3-phenyl propyl]piperidinium chloride; or
3-(2-hydroxy-5-methylphenyl)-N,N,N-triallyl-3-phenylpropan-1-aminium bromide.

24. A pharmaceutical composition comprising a therapeutically effective amount of a quaternary ammonium compound according to claim 1, and a suitable pharmaceutical carrier therefor.

25. A method of treating asthma in a mammal, said method comprising administering to said mammal, in need of such a treatment, a therapeutically effective amount of a quaternary ammonium compound according to claim 1.

26. A method of treating chronic obstructive pulmonary disease (COPD) in a mammal, said method comprising administering to said mammal, in need of such a treatment, a therapeutically effective amount of a quaternary ammonium compound according to claim 1.

27. A method of treating allergic rhinitis in a mammal, said method comprising administering to said mammal, in need of such a treatment, a therapeutically effective amount of a quaternary ammonium compound according to claim 1.

28. A method of treating urinary disorder in a mammal, said method comprising administering to said mammal, in need of such a treatment, a therapeutically effective amount of a quaternary ammonium compound according to claim 1.

29. A method of treating rhinorrhea due to the common cold in a mammal, said method comprising administering to said mammal, in need of such a treatment, a therapeutically effective amount of a quaternary ammonium compound according to claim 1.

30. A method of claim 25 wherein said mammal is a human.

31. A method of claim 26 wherein said mammal is a human.

32. A method of claim 27 wherein said mammal is a human.

33. A method of claim 28 wherein said mammal is a human.

34. A method of claim 29 wherein said mammal is a human
